(19) United States
(12) Patent Application Publication
(10) Pub. No.: US 2010/0071629 A1
(43) Pub. Date: Mar. 25, 2010

(54) MULTI-LAYER AQUACULTURE SYSTEMS CONSTRUCTED BY THE USAGE OF COMMERCIAL SUPPORTING STRUCTURES

(76) Inventors: Jiing-Kae Wu, Yung-Kang City (TW); Shyh-Min Hsiao, Kaohsiung City (TW)

Correspondence Address: Muncy, Geissler, Olds & Lowe, PLLC, P.O. BOX 1364, FAIRFAX, VA, 22038-1364, US

(21) Appl. No.: 12/564,588

(22) Filed: Sep. 22, 2009

(30) Foreign Application Priority Data
Sep. 23, 2008 (TW) 097136537

(51) Int. Cl. A01K 61/00; A01K 63/00; A01H 13/00

(52) U.S. Cl. 119/207; 119/245; 119/234; 47/1.4

(57) ABSTRACT

The present invention discloses a structure for supporting multi-layer aquaculture facilities by the usage of construction scaffold systems and other supporting structures used by commercial storage/retrieval and horticulture greenhouse systems, allowing rapid construction of multi-layer aquaculture systems to be achieved with commercially available components. The modifications and conversions of the components involve transforming existing supporting systems, stairways or powered elevating systems, environment adjusting modules, and positioning parts into various components of a multi-layer aquaculture facility, including: 1) various frames supporting water tanks and pipe lines in multi-layer aquaculture facilities, 2) vertical stairways and elevating systems for transporting people and goods in multi-layer aquaculture facilities, 3) environment adjusting modules, 4) holding, positioning and stabilizing components in multi-layer aquaculture facilities, and 5) personnel safety components in multi-layer aquaculture facilities. These aquaculture facilities include small aquaculture facilities used in laboratories and classrooms, medium-size aquaculture facilities used for exhibit and display purposes, and large aquaculture facilities used in aquaculture farms. The cultured aquatics include fishes, shrimps, shellfishes, and algae.

### TECHNICAL FIELD

The present invention relates to the construction of multi-layer aquaculture systems via commercial supporting structures, for building multi-layer aquaculture systems rapidly. The systems include small aquaculture facilities used in laboratories and classrooms, medium-sized facilities used for display purposes, and large facilities used in aquaculture farms.

### BACKGROUND

The traditional aquaculture industry generally utilizes lands and ponds with natural water supply as the culture environment for growing aquatic animals (fishes, shrimps and shellfishes etc.). However, due to lack of sufficient control, the culture practices within these environments may bring pollution to the surrounding ecosystem. In order to improve the aforementioned situation, some new culturists have adopted the factory style culture method; utilizing water recirculation and filtration techniques to cut down wastewater discharge, to minimize depletion of natural resources and to lower damages toward the natural environment. In addition, the factory style culture method also provides protection methods to reduce the attacks of pathogens towards the cultured organisms.

However, building these factory style culture systems requires higher technologies and more materials than building regular ponds, therefore a production increase over each unit of factory floor area is critical for balancing the added costs and for ensuring its commercial value.

An effective way to increase the production of unit factory floor area is to construct multi-layer cultivation troughs via the usage of support frames. In order to achieve this objective, various high density culture equipments have been developed, such as Taiwan Patent No. M243017 and Taiwan Patent No. 466905. Patent No. M243017 also disclosed an aquaculture device to be placed within waters; the aquaculture device includes a frame structure with a plurality of supporting components, and a plurality of culture areas coupled to the supporting components. The frame structure is placed within the culture ponds to increase production space for the cultured aquatics, thereby increasing the culture density over the unit pond floor. Patent No. 466905 disclosed a high density cultivation tank for cultivating live benthos. It places a spiral-like supporting structure within the cultivation tank; the cultivation sheets attached on the spiral-like supporting structure surround the tank's inner periphery; a water inlet pipe is placed at the side of the cultivation tank, where there are a number of water intake holes; at the bottom of the cultivation tank there is a water discharge outlet.

However, the methods mentioned above lack modular components required for rapid assembling of mass production facilities, thereby have following deficiencies: (1) supplies of supporting frames and other components are not in stock, (2) supplies of supporting frames and other components are not modularized, (3) most components require to be individually ordered and custom made, (4) supply of most components is not only time-consuming but also expensive, making it difficult for rapid construction of multi-layer aquaculture facilities for producing aquatic organisms in quantities.

On the other hand, Taiwan Patent No. M289024 discloses an indoor aquaculture device that allows for transportation, rapid installation, dismantling, and convenient operation such as feeding. Main body of the device is composed of frames and waterproof clothes, whereby the frames include two cultivation tanks and their top covers, and the waterproof clothes include liners and cover layers. The two cultivation tanks are placed on ground in a parallel position with a walkway space in between. The liners lining each cultivation tank to form cultivation ponds' water containment for aquatics living in. Although the device has the advantages of easy transportation and installation, it requires the buyers to order customized components for supporting cultivating tanks in order to make the use of it for improving the farm land usage (i.e. utilizing multi-layer cultivation design), thereby usage of the device in aquaculture facilities may involve high costs and a length of construction time.

To avoid the aforementioned situations and to coordinate with new development of multi-layer factory style culture method, the present invention provides a structure that allows for rapid construction of multi-layer aquaculture systems with the advantages of components standardizing and modularizing (making it easier and faster to obtain), and cost lowering to achieve the goal of building a multi-layer aquaculture system efficiently.

### SUMMARY OF THE INVENTION

An advantage of the present invention is to provide a multi-layer aquaculture system whereby the components are standardized.

Another advantage of the present invention is to provide a multi-layer aquaculture system whereby the components are modularized.

Yet another advantage of the present invention is to provide a multi-layer aquaculture system that utilizes components common to the commercial supporting structure, whereby the components have the advantages of being readily available, low cost, and modularized with a variety of parts.

The present invention discloses a multi-layer aquaculture system that utilizes commercial supporting structures such as construction scaffold systems, warehouse storage/retrieval supporting modules and horticulture greenhouse frames and accessories. The multi-layer aquaculture system comprises a plurality of vertical and horizontal rods where both ends of the horizontal rods are connected to vertical rods, forming “H” shapes; a plurality of intersecting rods, where both ends of the intersecting rods are connected to the plurality of vertical rods, forming “IxI” shapes; a plurality of deck boards clipped onto the horizontal rods via the parallel hooks located at both ends of the deck boards; a plurality of right-angled fixtures screw-locked onto the deck boards; at least one aquaculture tank installed on the deck boards and may be positioned through the plurality of right-angled fixtures; and a life support system, comprising a set of water inlet pipes placed on the deck boards via fixtures and connected to the aquaculture tank; a water pump for delivering water to the aquaculture tank via the set of water inlet pipes; a physical filtration tank for removing excrements, excess feeds and particles; and a nitrifying filtration tank to convert dissolved nitrogenous wastes into harmless nitrates.

It should be noted that the bearing capacity of construction scaffold systems and the warehouse storage/retrieval supporting modules and horticulture greenhouse frames and accessories are often lower than standard concrete buildings, thus the preexistence of shallow water cultivation technique is required to reduce the water weight in culture tanks used in multi-layer aquaculture systems. Aquaculture application of the multi-layer system mentioned in the present invention has been filed by the 2^{nd} inventor on Oct. 28, 2004 and the patent (U.S. Pat. No. 7,174,850) has been granted on Feb. 13, 2007, thus details of the multi-layer aquaculture system is not included in the present invention.

### BRIEF DESCRIPTION OF THE DRAWINGS

The present invention may be understood by the detailed descriptions of some preferred embodiments outlined in the specification and the drawings attached. However, it should be appreciated that all the preferred embodiments of the invention are for illustration, and not for limiting the scope of the present invention, wherein:

FIG. 1 illustrates the front view of a laboratory type multi-layer aquaculture facility constructed by the modifications and conversions of commercial supporting structures according to the present invention;

FIG. 2 is an illustration of the multi-layer shallow water aquaculture display facility according to the present invention;

FIG. 3 is an illustration of an aquatic organism display design “mother ship”, which is constructed by additions of decorative items over the multi-layer aquaculture display facility according to the present invention;

FIG. 4 illustrates the side view of the preferred embodiment for the multi-layer aquaculture systems with life-support devices, which has been designed according to the present invention.

### DETAILED DESCRIPTION

Some preferred embodiments of the present invention are described in greater detail. However, it should be recognized that the present invention can be practiced in a wide range of other embodiments besides those explicitly described, and the scope of the present invention is not expressly limited except as specified in the accompanying claims.

It should be noted that, portions of the diagrams are not shown to scale. Some dimensions of the related components are exaggerated and insignificant portions are not drawn to provide clearer description and comprehension of the present invention.

The present invention provides a structure for supporting multi-layer aquaculture systems by the usage of construction scaffold systems and warehouse storage/retrieval supporting modules and horticulture greenhouse frames and accessories, allowing rapid constructions of multi-layer aquaculture systems to be achieved. For this goal to be fully implemented, some modifications and conversions of existing parts and components are necessary. Such modifications and conversions involve transformation of existing vertical and horizontal rods, stairways or powered elevating systems, different types of environment adjusting modules, and modifications of positioning bars into various supports for multi-layer aquaculture systems and facilities, including: 1) various support frames for holding a variety of water tanks and pipe lines, 2) stairways and elevating systems for transporting people and goods, 3) various types of environment adjusting modules, 4) holding, positioning and stabilizing components, and 5) personnel safety components.

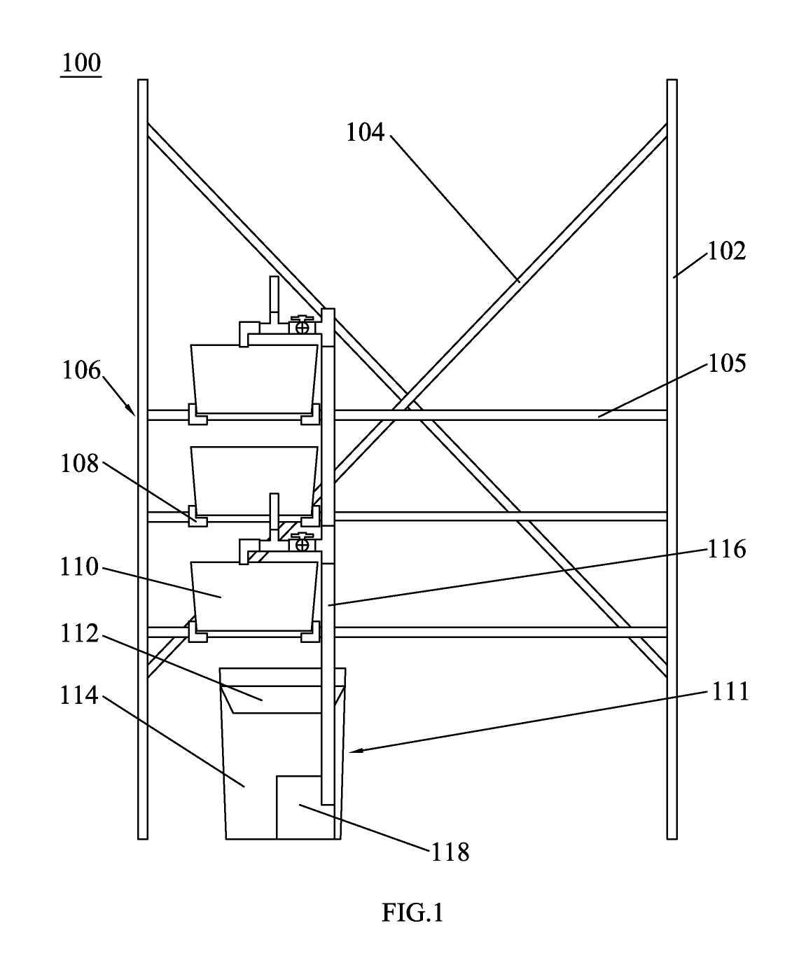

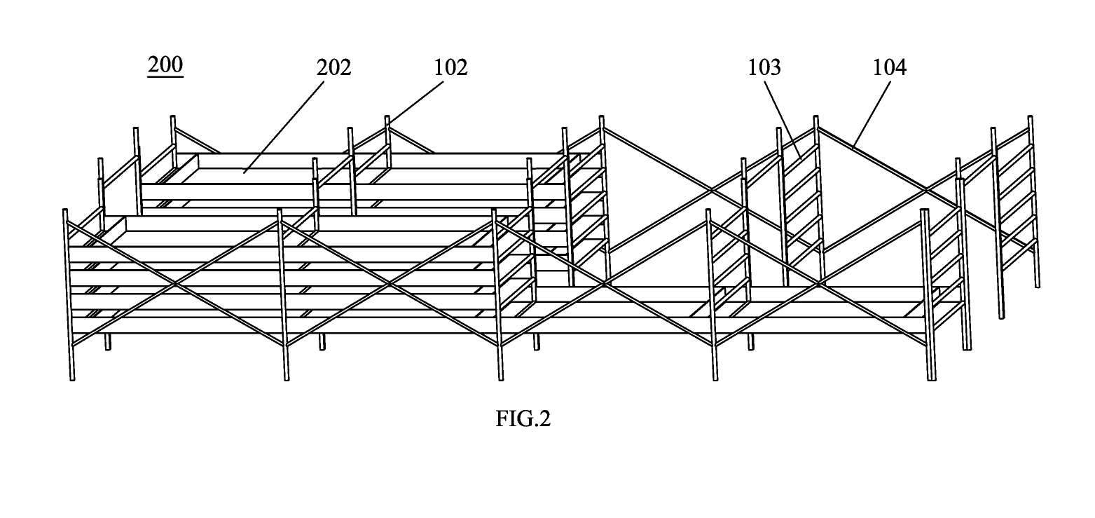

Referring to FIG. 1, it illustrates the front view of a (laboratory type) multi-layer aquaculture system 100 constructed by the modifications and conversions of commercial supporting structures according to the present invention. The multi-layer aquaculture facility 100 comprises multi-layer aquaculture support frames and an aquaculture system. The support frames include a plurality of vertical rods 102, a plurality of horizontal rods 103 (shown in FIG. 2), a plurality of intersecting rods 104, a plurality of deck boards 105, with parallel hooks 106 located at both ends of deck boards 105, and a plurality of right-angled fixtures 108. Parallel hooks 106 are hook structures at both sides of each deck board 105 to act as a safety device preventing accidental disengagements. The aquaculture system includes a plurality of aquaculture tanks 110 and a life support system 111. The life support system 111 comprises a physical filtration tank 112, a nitrifying filtration tank 114, a set of water inlet pipes 116 and a water pump 118.

As shown in FIG. 1, the multi-layer aquaculture support frames are constructed with horizontal rods 103 (shown in FIG. 2) soldered to a plurality of vertical rods 102 forming “H” shapes, and a plurality of intersecting rods 104 are connected to a plurality of vertical rods 102, forming “IxI” shapes. Deck boards 105 are positioned onto the support frames with their parallel hooks 106 (located at both ends of deck boards 105) clipped onto horizontal rods 103. Right-angled fixtures 108, fixed onto deck boards 105 via screw-locking, serving as positioning devices for aquaculture tank 110 on deck boards 105. The set of water inlet pipes 116 taking water away from a life support system 111 are secured on deck boards 105 via fixtures (not shown), its branches deliver water to each aquaculture tank 110. Cultivation water is then pumped into each aquaculture tank 110 by a water pump 118 through the set of water inlet pipes 116, after the water has been processed through physical filtration tank 112 and nitrifying filtration tank 114 for removing particles and dissolved wastes.

Referring to FIG. 2, it illustrates the multi-layer shallow water aquaculture display facility 200 according to the present invention. As illustrated, in multi-layer shallow water aquaculture display facility 200, both ends of a plurality of horizontal rods 103 are connected to a plurality of vertical rods 102, forming “H” shape horizontal support, and both ends of a plurality of intersecting rods 104 are connected to a plurality of vertical rods 102, forming “IxI” shapes, to obtain a stabilizing effect. It should be noted that, deck boards 105 with parallel hooks 106, right-angled fixtures 108 and aquaculture tank 110 shown in FIG. 1 have been transformed to be shallow water aquaculture tanks 202. Shallow water aquaculture tanks 202 are installed directly over horizontal rods 103, with non-slip fixtures (not shown) hooked on horizontal rods 103, to prevent unwanted disengagement. Moreover, besides shallow water aquaculture tanks 202, other components of multiple layer water aquaculture systems such as water delivery pipes, water outlet pipes, and life support systems etc. (not shown in FIG. 2) are supported through the same frame structure.

During the actual implementation, the commercial supporting structures utilized by the present invention include stairways and powered elevation for workers and materials, plus personnel safety components, etc. are utilized for the convenience of moving aquatics, facility setting up, and to ensure the safety of workers. In addition, anti-vibration components may be installed on the scaffold systems to further improve stability of the frames. Moreover, the aforementioned vertical rods, horizontal rods, intersecting rods, deck boards and parallel hooks are all commercially available components that require only minor modifications.

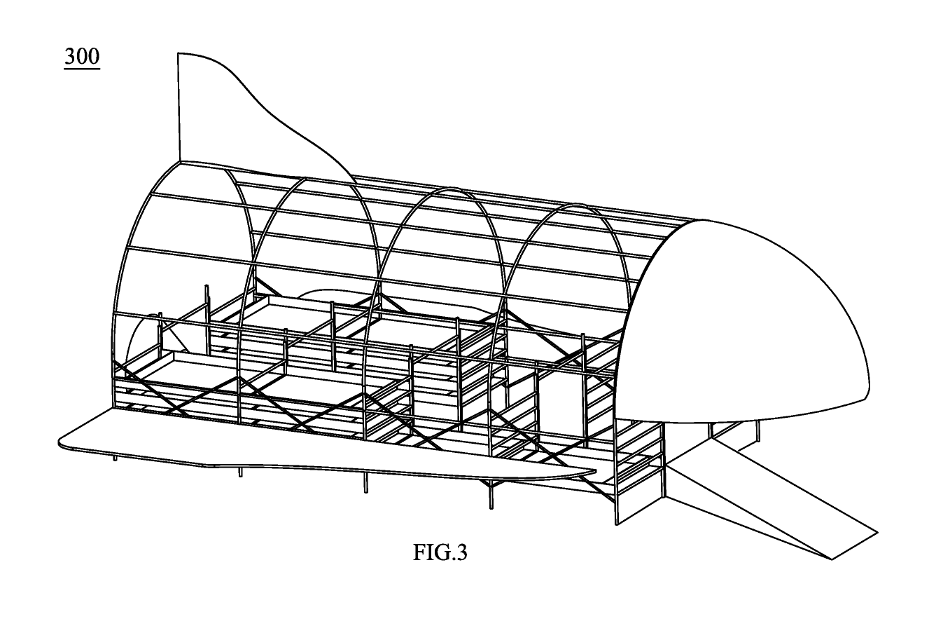

FIG. 3 shows an aquatic organism display design, ‘mother ship’ 300 that has a multi-layer shallow water aquaculture system built-in. The frame structure of the ship is arranged according to the present invention. For dressing the display mother ship 300 as a space craft, multi-layer shallow water aquaculture system 200 of FIG. 2 is partially covered with additional decorative items. On top of the artistic display, by utilizing commercially available supporting components, construction of the display mother ship 300 also has the advantages of rapid installation/dismantling and easy transportation.

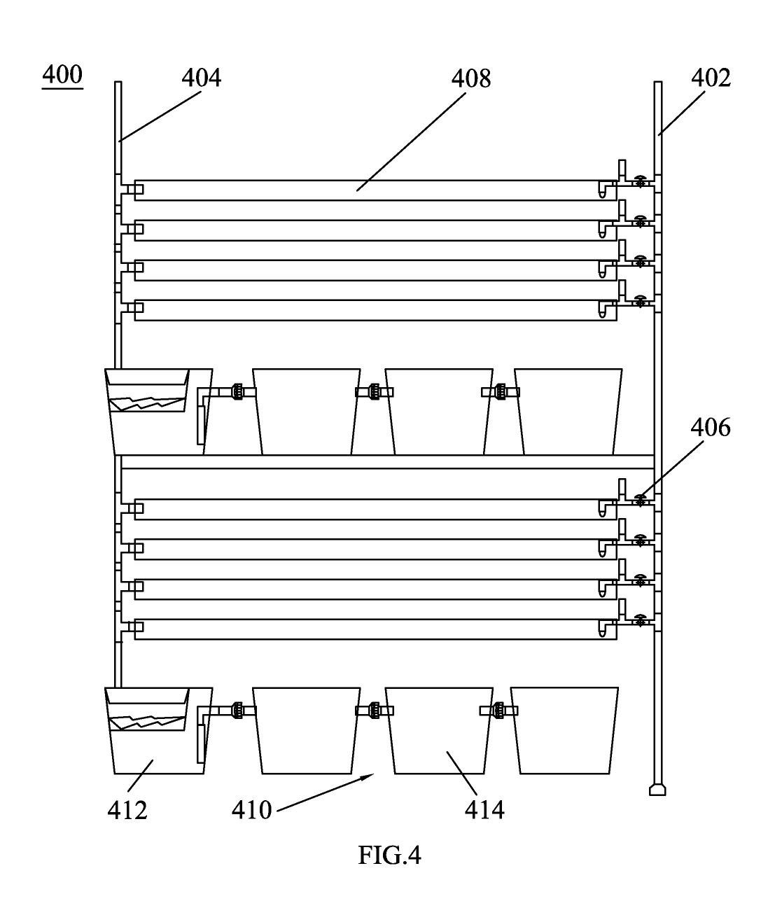

Referring to FIG. 4, it illustrates the attached piping and life-support tanks of a preferred embodiment of the multi-layer shallow water aquaculture facility 400, which has been designed according to the present invention. The vertical pipes located at the right side of the multi-layer shallow water aquaculture system 400 is water delivery pipe 402, while the vertical pipe located on the left side of multi-layer shallow water aquaculture facility 400 is water outlet pipe 404. The water flow coming from water delivery pipe 402 is adjusted by water pressure adjuster 406, and is then flow through each layer of shallow water aquaculture tanks 408. Water outlet pipe 404 collects the water from each layer of shallow water aquaculture tanks 408 and sends the water back into life support system 410 (arranged in series). In order to utilize the three-dimensional space provided by the scaffold frames, life support system 410 may be installed on various layers, wherein at least one physical filtration tank 412 and a plurality of nitrifying filtration tank 414 are in place. The water flows out of shallow water aquaculture tanks 408 through water outlet pipe 404 out of shallow water aquaculture tanks 408 first goes through a physical filtration tank 412 to have its contents of digestive waste and excess feeds removed and then goes through nitrifying filtration tanks 414 for converting its contents of nitrogenous metabolic wastes into harmless nitrates.

The present invention may be applied to live aquatics include various types of fishes, shrimps, crabs, shellfishes (including oysters and abalones), and algae (including Gracilaria), and the aforementioned shrimps include, but not limited to white shrimp (Litopenaeus vannamei), tiger shrimp (Penaeus monodon), blue shrimp (Penaeus setiferus), or Kuruma shrimp (Penaeus japonicus).

From the aforementioned descriptions, it can be seen that the present invention offer the following advantages: 1) a structure that allows for rapid construction of multi-layer aquaculture systems; 2) components and parts that are standardized and modularized (making it easier and faster to obtain), leading to lower production costs, and achieving high-efficiencies on building a multi-layer aquaculture facility.

The foregoing descriptions are preferred embodiments of the present invention. As is understood by a person skilled in the art, the aforementioned preferred embodiments of the present invention are illustrative of the present invention rather than limiting the present invention. The present invention is intended to cover various modifications and similar arrangements included within the spirit and scope of the appended claims, the scope of which should be accorded the broadest interpretation so as to encompass all such modifications and similar structures.

What is claimed is:

1. A multi-layer aquaculture system constructed by the usage of commercial supporting structures, comprising:
a plurality of vertical rods;
a plurality of horizontal rods, whereby both ends of said horizontal rods are connected to said plurality of vertical rods forming “H” shapes;
a plurality of intersecting rods, whereby both ends of said intersecting rods are connected to said plurality of vertical rods forming “IxI” shapes;
a plurality of deck boards, clipped onto said horizontal rods via parallel hooks located at ends of said deck boards;
a plurality of frames, clipped onto said horizontal rods via parallel hooks located at ends of said deck boards;
at least an aquaculture tank, positioned or installed on said deck boards or said plurality of frames; and
a life support system, comprising:
a set of water inlet pipes, positioned over said deck boards via fixtures and connected to said aquaculture tank;
a water pump, for the delivery of water used for cultivation to said aquaculture tank via said set of water inlet pipes;
a set of water outlet pipes, connected to said aquaculture tank, for directing water from said aquaculture tank;
a physical filtration tank, connected to said set of water outlet pipes, for removing excrements and excess feeds in culture water; and
a plurality of nitrifying filtration tanks, connected to said physical filtration tank, for converting its contents of metabolic wastes into nitrates.

2. The multi-layer aquaculture system constructed by the usage of commercial supporting structures of claim 1, wherein said deck boards, said parallel hooks, said frames and said aquaculture tank may be replaced by a shallow water aquaculture tank installed directly over said horizontal rods.

3. The multi-layer aquaculture system constructed by the usage of commercial supporting structures of claim 2, wherein non-slip fixtures are located at the connected regions of said shallow water aquaculture tank and said horizontal rods.

4. The multi-layer aquaculture system constructed by the usage of commercial supporting structures of claim 1, further comprising installation of stairways or powered elevating systems for transporting people and goods on said multi-layer aquaculture system.

5. The multi-layer aquaculture system constructed by the usage of commercial supporting structures of claim 1, further comprising installation of personnel safety components on said multi-layer aquaculture system.

6. The multi-layer aquaculture system constructed by the usage of commercial supporting structures of claim 1, further comprising installation of anti-vibration components on said multi-layer aquaculture system.

7. The multi-layer aquaculture system constructed by the usage of commercial supporting structures of claim 1, further comprising decorative items added over said multi-layer aquaculture system.

8. The multi-layer aquaculture system constructed by the usage of commercial supporting structures of claim 1, further comprising addition of various environment adjusting modules on said multi-layer aquaculture system.

9. The multi-layer aquaculture system constructed by the usage of commercial supporting structures of claim 1, wherein said aquaculture organisms include various types of fishes, shrimps, crabs, shellfishes, and algae.

10. The multi-layer aquaculture system constructed by the usage of commercial supporting structures of claim 9, wherein said shrimps include, but not limited to white shrimp (Litopenaeus vannamei), tiger shrimp (Penaeus monodon), blue shrimp (Penaeus setiferus), or Kuruma shrimp (Penaeus japonicus).

11. The multi-layer aquaculture system constructed by the usage of commercial supporting structures of claim 9, wherein said shellfishes include oyster or abalone.

12. The multi-layer aquaculture system constructed by the usage of commercial supporting structures of claim 9, wherein said algae include Gracilaria.

13. The multi-layer aquaculture system constructed by the usage of commercial supporting structures of claim 1, wherein said commercial supporting structures include construction scaffold systems.

14. The multi-layer aquaculture system constructed by the usage of commercial supporting structures of claim 1, wherein said multi-layer aquaculture system include supporting modules utilized by storage/retrieval systems.

15. The multi-layer aquaculture system constructed by the usage of commercial supporting structures of claim 1, wherein said multi-layer aquaculture system include frames and accessories utilized by horticulture greenhouse systems.
